(19) United States
(12) Patent Application Publication
(10) Pub. No.: US 2022/0048034 A1
(43) Pub. Date: Feb. 17, 2022

(54) ARRANGEMENT FOR IMPLEMENTATION OF IN VITRO BIOCOMPATIBILITY TESTS

(71) Applicant: Fraunhofer-Gesellschaft Zur Foerderung Der Angewandten Forschung E.V., Muenchen (DE)

(72) Inventors: Constantin ISSLEIB, Dresden (DE); Juliane SPOH, Dresden (DE); Susanne KURZ, Dresden (DE)

(73) Assignee: Fraunhofer-Gesellschaft Zur Foerderung Der Angewandten Forschung E.V., Muenchen (DE)

(21) Appl. No.: 17/274,496

(22) PCT Filed: Sep. 12, 2019
(86) PCT No.: PCT/EP2019/074319

§ 371 Date: Mar. 9, 2021

(30) Foreign Application Priority Data
Sep. 19, 2018 (DE) 10 2018 215 956.4
Dec. 11, 2018 (DE) 10 2018 221 415.8

(51) Int. Cl. B01L 9/00; C12M 1/32; C12M 1/00; C12M 3/00

(52) U.S. Cl. CPC B01L 9/52; C12M 23/12; C12M 23/38; C12M 23/22; C12M 23/44; B01L 2300/0829; B01L 2300/043; B01L 2300/0822; B01L 2200/028; B01L 2300/163; B01L 2200/0689

(57) ABSTRACT

An assembly for performing in vitro biocompatibility tests, at least one sample is arranged on a surface of a base plate or the sample forms a surface or a surface region of the base plate. A holding element having at least one through-hole is placed onto the sample so that a first opening of the through-hole, which first opening is arranged facing the base plate, is arranged in the region of the sample. The through-hole, with the hollow space thereof, and the sample form a cavity. A cover element is placed onto and fastened on the holding element so that a compressive force acts on the holding element, which compressive force leads to at least partial deformation of the holding element and to a fluid-tight closure of the first opening of the through-hole, which first opening is arranged facing the base plate.

### BACKGROUND OF THE INVENTION

The invention relates to an arrangement for the implementation of in vitro biocompatibility tests for preferably different, at least one-sided planar, impermeable material samples which can be examined with a liquid or solution in which biological material is contained. Of particular interest are tests on materials which can be used in medical technology. These can be for example materials for implants or coated surfaces in cell culture and for diagnostic tests.

The biological evaluation of solid, solvent-impermeable test materials takes place basically, according to the current state of the art, within a solution. This means that a test piece/test material is placed in a microtitre plate and is embedded in a corresponding solution (e.g. cell culture medium) and the solution can consequently completely surround the test piece.

If the entire test piece is observed, the result between various materials or also between various manufacturers is crucial differences in the specific surface, caused by the size and height of the test pieces. Even within the same material, it is technically highly challenging to produce two identical material samples. In addition, a test piece generally has only one “good” side which represents the surface properties of the material. The described differences and influential factors have effects on quantifiable, biological results.

There is a lack of a standardised, non-complex methodology which offers exclusively a defined surface on the good side of a sample for biological evaluation, produces a base-volume ratio within a cavity, which is suitable for cell culture tests, and at the same time is easy to handle in a sterile manner and ensures that this defined surface can be conferred at the same time on further test pieces.

In the case of the biological assessment of materials in the medical product context, there are currently clear instructions given by the standard DIN EN ISO 10993. It is described therein that a test piece/test material is placed in a microtitre plate and is embedded in a solution and relevant test methods are implemented in the total volume of the solution. Since this standard prescribes the minimum amount of testing, it has not been necessary to date, from the point of view of the manufacturer/testing laboratory, to expand the test batch.

The invention standardises the testing process, in which, for each test piece, the same surface and the same volume of liquid is offered to a solution and the biological material only obtains contact with the solution to the good side of the test piece. Biological results, even between materials, are hence quantifiable and comparisons are significantly more exact.

In biological test scenarios, frequently microtitre plates are used. There are various formats, such as e.g. 6-well, 12-well, 24-well and 96-well plates. The wells (cavities) of a microtitre plate are all of the same size. Normally, the smallest possible well size is selected into which the sample to be tested fits. It is consequently disadvantageous with the microtitre plates that different sample geometries or samples with the same geometry, but with slight size differences, are tested in the constant well format since the result hereby is a high, multifactorial error quota. The interaction of the biological material with the surrounding microtitre plate consisting of plastic is in addition entirely neglected.

Thus adhesive solutions are also known in which cavities with a depth up to 2.5 mm are present. The adhesive effect is released thereby when pressing in a sample. They are used only on comparatively smooth surfaces and frequently for cell-microscopic purposes. For the simple reason of the low depth of the cavity, this solution is not suitable for our purposes of material testing since it does not offer an adequate base-volume ratio for cell culture batches. A significant disadvantage could in addition be that the adhesive is transferred into the cell culture medium and possibly has a cell-toxic effect or interacts with dissolved, biological material. Furthermore, adhesive residues could remain adhering on the material sample after use so that the sample must be cleaned subsequently.

In addition to adhesive- and pressure solutions, there is the physical barrier solution (hydrophobic barrier) which is based on the generation of hydrophobic properties in the boundary region. These hydrophobic regions can have a negative influence on the general growth and cultivation of cells on the material surfaces to be tested. Further disadvantages of these barrier solutions are too small a height of the cavities and a general suitability for only comparatively smooth surfaces.

Published pressure solutions allow up to 100 μl volumes to be received in a cavity. For example a screw closure presses elastic material onto a glass object carrier. These configurations are intended for small volumes on glass object carriers. On samples, in particular with a different geometry and size, sufficiently flexible reactions are not possible. In addition, multiday cell culture tests in these small volumes and without ensuring a gas exchange are not possible.

All indicated solutions were developed for glass object carriers or comparatively smooth surfaces and are inter alia unsuitable for other surfaces, in particular because of different roughnesses. In particular the hydrophobic barrier solution could significantly lose impermeability from time to time with rough surfaces. In addition, all cited solutions with simultaneous processing of a plurality of test pieces are very labour-intensive. In part, the cited solutions also lack simple, sterile handling capacity of the test pieces with a produced cavity and an extra “packaging” in Petri dishes, or the like, becomes necessary.

### SUMMARY OF THE INVENTION

It is therefore the object of the invention to indicate possibilities for tests on samples when implementing in vitro biocompatibility tests, in which a standardised surface is produced on test pieces of different roughnesses, a less labour-intensive production of these cavities, comparing a plurality of test pieces, is even possible and a simple, sterile handling capacity of the test pieces with the produced cavities is ensured.

According to the invention, this object is achieved with an arrangement which has the features of the claims.

At least one sample is thereby disposed on a surface of a base plate. The at least one sample can however also form a surface or a surface region of the base plate. A receiving element having at least one through-hole can be placed preferably in an exact fit.

A receiving element having at least one through-hole can be placed on the at least one sample such that a first opening, which is disposed pointing in the direction of the base plate, of the at least one through-hole is disposed in the region of the at least one sample. The at least one sample and the at least one through-hole thereby form the hollow space of a cavity.

A cover element can be placed and fixed on the receiving element such that a pressure force acts on the receiving element, which pressure force leads to an at least partial deformation of the receiving element and the fluid-impermeable closure of the first opening, which is disposed pointing in the direction of the base plate, of the at least one through-hole. During a test, no liquid can hence emerge there.

The sample should be dimensioned such that, with its at least one-sided, planar side, it covers the edges of the at least one through-hole pointing in the direction of the base plate.

For equalisation of different sample heights, advantageously plate- or disc-shaped elements with correspondingly adapted thickness can be disposed in the region of one or more through-hole(s) on the surface of the base plate, which is disposed orientated towards the receiving element, between base plate and material sample. On such a plate or disc-shaped element, a sample can then be disposed, as was described previously. Hence in particular height differences can be compensated for. The insertion of positioning aids for simple orientation of samples on the surface of the base plate is possible.

The hollow space of the respective at least one through-hole of the receiving element and the sample should thereby form at least a 5 mm high cavity (undeformed state), wherein the inner surface area of the through-hole should form the side wall and the sample should form the base of the cavity.

Suitable through-holes for the receiving element can be present on the cover element, via which the respective cavity can be filled with a liquid in which for example living cells can be contained. The cover element can be provided with markings for easier assignment of the through-holes.

Advantageously, base plate and cover element are connected together on one side, preferably articulated, and can in addition be exchangeable. Thus, the cover element can be folded up and down in order to insert the arrangement and the tests can be implemented in the closed state. When placing the receiving element between base plate and cover plate, the pressure forces can be exerted for example by means of a click-lock mechanism as example of a form-fit connection. Hence, adhesive materials, such as e.g. glues, can be dispensed with.

On the base plate and the cover element, closing elements should be configured, with which a form-fit connection of base plate and cover element and also a pressure force exertion on the receiving element are achievable.

The closing elements and a connection to the base plate and cover element should advantageously be connected together and thereby the corresponding elements should be disposed on oppositely-disposed sides of the arrangement. Thus at least one joint, for example a film- or hinge joint can be disposed on one side and the closing elements on an opposite side.

Because of this type of connection of base plate and cover element, additional elements, with which closure of an arrangement can be achieved and which must be fitted as individual parts on the arrangement, can be dispensed with. Thus for example screw connections are critical because of their handling capacity. In addition, additional elements make sterilisation difficult even if they need not be exchanged after each implemented test. In the case of the arrangement according to the invention, advantageously no additional individually manageable connection elements with which the base plate must be connected to a cover plate are required.

In order to facilitate closure and opening of the arrangement, a handle can be present.

The receiving element can be formed advantageously from or with an elastically deformable material which preferably has a Shore hardness in the range of 30 to 50. It can therefore be formed completely from deformable material, particularly preferably from an elastically deformable material, or be provided with elastically deformable material as composite material, at least on the surface which abuts on the base plate or at least one sample. As a result, additional sealing elements which must be fixed in a form-fit and/or frictionally and which likewise would be disadvantageous during a sterilisation can be dispensed with.

On the cover element, at least one opening should be disposed such that it leaves at least partially open a second opening of the respective through-hole of the receiving element which is disposed in the direction of the cover element.

A closing cover can also be placed on the cover element and thereby, preferably for a defined prescribable positioning and/or orientation on a surface of the closing cover and/or cover element, at least one construction element can be present for a form-fit orientation.

A preferably at least partially optically transparent closing cover can be placed on the fitted and closed arrangement. Hence closing of the cover element openings and hence the sterile handling capacity of the closed arrangement can be achieved outside a sterile workbench. The closing cover should thereby be able to be placed in a prescribed orientation and enable a gas exchange between environment and cavity content.

Cover element and receiving element can have through-holes which correspond to the current well formats and are exchangeable modularly. This also applies to the cover element and the receiving element adapted thereto.

Elements which enable a gas exchange with the ambient atmosphere can be present on a cover element. This can be at least one opening. An opening can also be provided with a membrane which should be gas-permeable. A membrane can thereby be selectively permeable advantageously also for at least one selected gas or gas mixture.

The receiving element should be retained in a form-fit in the base plate and/or the cover element. As a result, a defined positioned fixing of through-holes with respect to the arrangement of respective samples can be achieved.

A plurality of through-holes can be configured or disposed in assigned groups. Thus, according to the size of the sample, a group of through-holes can be used in one receiving element for respectively one sample. On a plurality of samples, the same groups of cavities can be produced. This makes it possible to implement multiple assays on one sample and multiple assays over a plurality of samples. Likewise different tests can be implemented in parallel on one sample.

Also a plurality of different samples can be disposed on one surface of a base plate or the different samples can form respectively a surface or a surface region of the base plate.

On the base plate, at least one opening can be present for guiding objects, in particular object carriers, in and out. By means of such an opening, objects can be inserted specifically into the arrangement from outside, moved therein and removed again. At least one edge present on the opening can facilitate guiding an object into the arrangement or out of the arrangement.

As standardised surfaces, the microtitre plate formats which are normal in molecular-/cell biology have been chosen to date. Microtitre plates use small, defined, round cavities in order to implement molecular-biological tests as standard. Hence a very good reference point or comparison to the already established biological evaluation methods in the laboratory is present. Advantageously, receiving elements, the format of which corresponds to conventional microtitre plates or approximates to them, can be used in the invention.

With the invention, a limited, water-impermeable space with a standardised surface area with at least one through-hole can be made available for at least one sample or test piece. The height of the resulting cavities is 5 mm. By producing standardised or the same surfaces on the samples and the adapted liquid volume in these cavities, a biological evaluation of samples can give far more precise information than the state of the art has allowed to date. Edges, walls and rear-sides of through-holes with the corresponding surfaces of base plate and cover element and hence concomitant error sources, with respect to quantifiable, biological results, can be systematically avoided by the invention.

Using a press-on solution requires no adhesives or similar substances which diffuse into the liquids in contact with solutions. In addition, the receiving element hence remains completely untreated since the elastic material leaves no residue behind on a sample or on a test piece. The receiving element preferably consists of a biocompatible and possibly also a chemically inert material so that interactions with solvents or biological material during the test time can be minimised. Furthermore, the invention can be configured to be reliably solvent-impermeable, even with rough surfaces, since the elastic material is pressed easily onto the surfaces.

The invention can be used in order to compare samples/test pieces of the most varied material compositions and dimensions in a biological context as standard. In particular the material testing in the field of medical technology (inter alia also implants) offers an extensive spectrum of material compositions and material properties for which such a comparison method, which provides representative and reproducible results, is urgently required.

With the invention, also biological tests can be implemented with samples with a coating made of a material, degradable materials and material coatings in which the described arrangement can be adapted in order to be able to ensure the relative fluid density during the test.

The biological evaluation of materials in routine laboratories can be used/offered by the invention. In the future also patient-specific reactions can be tested on various materials, as a result of which material compositions can be offered specifically for clients.

In biological and also material-scientific research, the invention offers a huge field of application since basically all materials which are impermeable for the time of the test can be examined biologically as standard. With the assistance of the arrangement according to the invention, the molecular-biological assays established for the microtitre plates can be transferred directly to material testing and, on the basis of the resulting quantitative measuring data, a direct comparison between material and microtitre plastic as reference can be implemented.

In the field of biological testing of medical products (ISO 10993), new quantitative methods could be used by using the invention which would not be possible without producing a standardised surface on the good side of a test piece.

### DESCRIPTION OF THE DRAWINGS

The invention is intended to be explained subsequently in more detail, by way of example.

There are thereby shown:

FIG. 1 consists of FIGS. 1A-1C and are an example of a base plate with a suitable receiving element and also a sample in perspective illustration;

FIG. 2 a perspective illustration of a base plate with inserted sample;

FIG. 3 a perspective illustration of a base plate with inserted sample and receiving element placed thereon;

FIG. 4 an example of a cover element which can be used in the arrangement according to the invention;

FIG. 5 an assembly drawing of an example of an arrangement according to the invention;

FIG. 6 an example of a closing cover and

FIG. 7 a closing cover which is placed on an arrangement consisting of base plate, sample with receiving element and cover element.

### DETAILED DESCRIPTION OF THE INVENTION

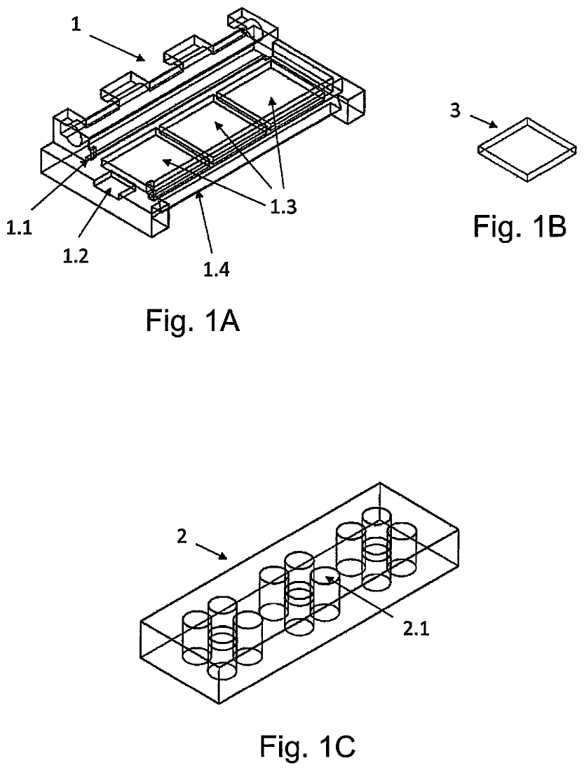

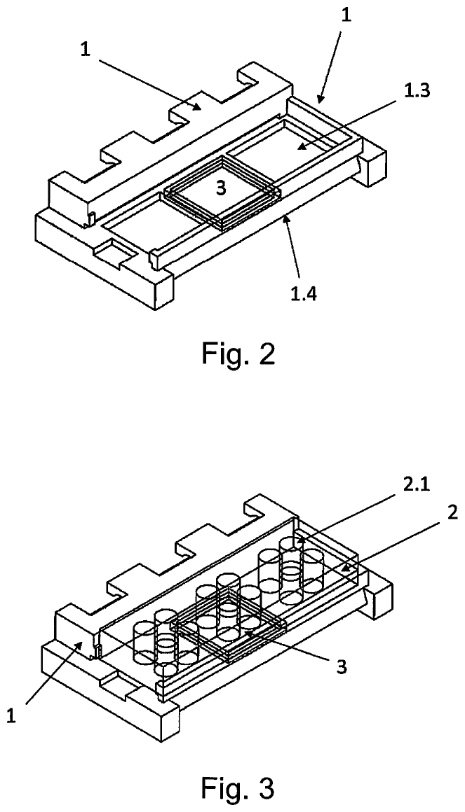

In FIG. 1A, an example of a base plate 1 is shown. Receiving means 1.3 for a form-fit hold of samples 3 are present in the base plate 1.

In addition, a gap-shaped opening 1.1 for introducing and removing objects, in particular object carriers, and an edge 1.2, which is intended to simplify this process, are present.

FIG. 1B shows an example of a sample 3 which can be placed on a surface of the base plate 1. This is effected preferably in the region of a receiving means 1.3.

After inserting at least one sample 3, a receiving element 2, shown in FIG. 1C, can be placed on the base plate 1. Through-holes 2.1 are present on the receiving element 2 and disposed such that they communicate with a surface region of a sample 3 and hence can form a cavity which can be filled with a liquid in which particularly biological material is contained in order to be able to test the respective sample 3 in biological or biochemical scenarios. This construction is shown in FIG. 3.

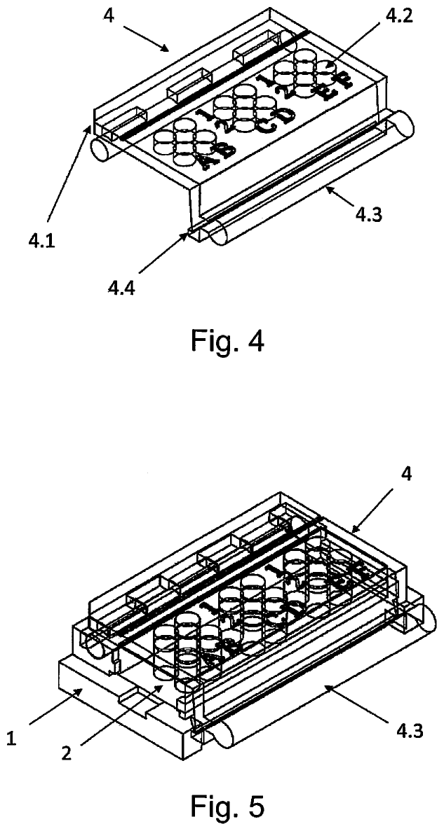

The cover element 4 shown in FIG. 4 can be connected, in an articulated manner, to the base plate 1 on an end-side. In the cover element 4, likewise through-holes 4.2 are present in this example and disposed such that they communicate with the samples 3 and the through-holes 2.1 in the receiving element 2 so that filling and removal of liquids in the through-holes 2.1 and a gas exchange is possible. In addition, the content of the cavities can be monitored visually. On the end-side of the cover element 4 which is disposed opposite the articulated connection, an edge 4.4 is configured, which edge, in the assembled state, can engage a lower edge 1.4 of the base plate 1 for a form-fit connection. As a result, pressure forces can be exerted on the elastically deformable receiving element 2 which effect pressing together and a fluid-impermeable closure of the cavities. The receiving element 2 is thereby elastically deformed. On this end-edge, a gripping element 4.3 with which opening and closing of the arrangement can be facilitated is present for convenience.

FIG. 5 shows an assembled closed state.

On the cover element 4, markings or characterising elements, for example in the form of letters and/or numbers, can be present, with which the assignment and recognition of specific samples 3 can be facilitated.

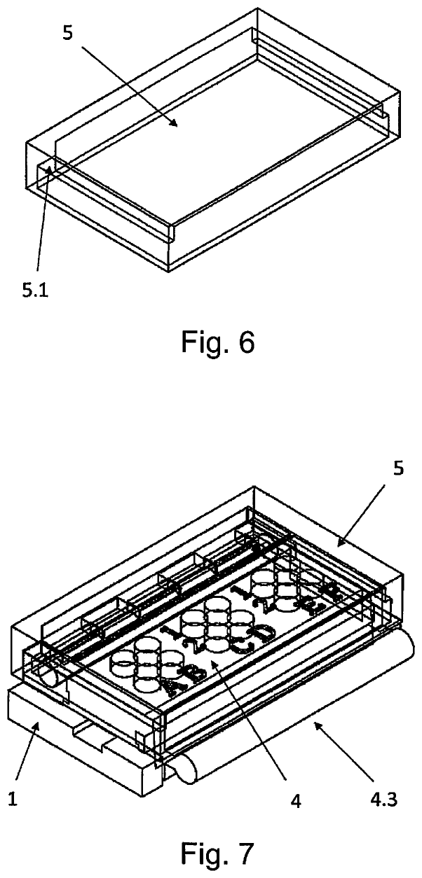

The closing cover 5 shown in FIG. 6 can be placed on the cover element 4 and be fixed on the cover element 4 and/or the base plate 1 in a form-fit, as is shown in FIG. 7. The closing cover 5 has, for this purpose, a circumferential edge 5.1 which enables, in cooperation with the edge 4.1 of the cover element 4, placing on the cover element 4 in a prescribed orientation.

The closing cover 5 should be optically transparent, at least in the region which covers the through-holes 2.1 and 4.2.

The receiving element 2 can consist for example of silicone and have a dimensioning, as was mentioned in the general part of the description.

The surface of the base plate 1 on which the receiving element 2 is placed has web-like raised portions on which outer edges of the receiving element 2 can abut and thus a form-fit hold of the receiving element 2 on the base plate 1 can be achieved.

Also on the cover element 4, web-like raised portions can be configured in the direction of the receiving element 2, with which a form-fit hold of the receiving element 2 can be achieved.

On the end-side, base plate 1 and cover element 4 can be connected together in an articulated manner so that the cover element 4 can be folded up and down. On the opposite end-side, closing elements can be configured on base plate 1 and cover element 4. They can form a kind of clip-on or snap-on closure when the cover element 4 closes the arrangement.

The closing elements can be configured, dimensioned and disposed such that, when closing the cover element 4 and the form-fit engagement of the closing elements between base plate 1 and cover element 4, the pressure forces act on the receiving element 2, with which the required deformation, which leads to closure of the openings of through-holes 2.1 which are disposed on the surface of the respective sample 3.

With pressure forces acting between base plate 1 and cover element 4, on the receiving element 2 and the surface of the sample(s) 3 orientated towards the receiving element 2, a fluid-impermeable seal between the surface of the receiving element 2 and the surface of the sample(s) 3, orientated towards the latter, can be achieved in the region of the cavities, with correspondingly deformed receiving element 2. For this purpose, the respective surface sizes of the sample(s) 3 in the region of the first openings and the configuration in this region should be dimensioned and chosen such that no liquid can emerge.

On the cover element 4, openings 4.2 are present, which are dimensioned and disposed such that second openings of through-holes 2.1 which are disposed on the cover element 4 are kept free so that a gas exchange with the environment can be effected. The cavities formed by the through-holes 2.1 and the sample(s) 3 can be filled via the second openings which are disposed on the surface of the receiving element 2 opposite the first openings, i.e. on the side orientated towards the cover element 4, with biological material and solvent before the respective test is implemented. During or after a test, liquid can be likewise removed through the through-holes 2.1 and 4.2 so that washing steps of the cavities are also possible.

Base plate 1, cover plate 4 and closing cover 5 are formed from autoclavable plastic (polycarbonate) in this example.

1. An arrangement for the implementation of in vitro biocompatibility tests, in which at least one sample is disposed on a surface of a base plate or in which the at least one sample forms a surface or a surface region of the base plate and a receiving element having at least one through-hole can be placed on the at least one sample such that a first opening, which is disposed pointing in the direction of the base plate, of the at least one through-hole is disposed in the region of the at least one sample,
the at least one through-hole with its hollow space and the sample forming a cavity, and
the receiving element being formed from or with an elastically deformable material;
a cover element placed and fixed on the receiving element such that a pressure force acts on the receiving element, which leads to an at least partial deformation of the receiving element and the fluid-impermeable closure of the first opening, which is disposed pointing in the direction of the base plate, of the at least one through-hole,
the base plate and the cover element being connected together on one side by at least one joint; the at least one joint and closing elements being arranged at oppositely-disposed sides of the arrangement.

2. The arrangement according to claim 1, wherein the receiving element is formed from a biocompatible and/or sterilisable material.

3. The arrangement according to claim 1, wherein the receiving element is formed from or with an elastically deformable material which has a Shore hardness in the range of 30 to 50.

4. The arrangement according to claim 1, wherein the at least one sample can be fixed in a form-fit on the surface of the base plate.

5. The arrangement according to claim 1, wherein at least one opening is disposed on the cover element such that it leaves at least partially open a second opening of the respective through-hole of the receiving element which is disposed in the direction of the cover element.

6. The arrangement according to claim 5, wherein on the base plate and the cover element, closing elements are positioned, with which a form-fit connection of base plate and cover element and also a pressure force exertion on the receiving element are achievable.

7-8. (canceled)

9. The arrangement according to claim 1, wherein corresponding to the current well formats in cell culture research, the cover element and the receiving element adapted thereto are modularly exchangeable.

10. The arrangement according to claim 1, wherein the at least one sample can be inserted in a cavity which is configured in the base plate and, after placing the receiving element, one or more through-holes are disposed in the region of a sample and the respective sample does not thereby contact the inner wall of the adjacent through-holes of the receiving element.

11. The arrangement according to claim 1, wherein a plurality of through-holes is configured or disposed in assigned groups.

12. The arrangement according to claim 1, wherein the receiving element is retained in a form-fit in the base plate or the cover element.

13. The arrangement according to claim 1, wherein a closing cover can be placed on the cover element, and allows a gas exchange with the environment, for a defined prescribable positioning or orientation on a surface of the closing cover or the cover element, at least one construction element is present for a form-fit orientation.

14. The arrangement according to claim 1, wherein on the base plate, at least one opening is present for guiding objects, in and out.

15. The arrangement according to claim 1, wherein elements which allow a gas exchange with the ambient atmosphere are present on the cover element or on a closing cover.

16. The arrangement according to claim 1, wherein a plurality of different samples are disposed on a surface of a base plate or various samples respectively form a surface or a surface region of the base plate.
